(19) United States
(12) Patent Application Publication
(10) Pub. No.: US 2021/0353151 A1
(43) Pub. Date: Nov. 18, 2021

(54) TARGETED FLUORESCENT MARKERS IN COMBINATION WITH A FLEXIBLE PROBE

(71) Applicant: On Target Laboratories, LLC, West Lafayette, IN (US)

(72) Inventors: Sumith A Kularatne, West Lafayette, IN (US); Benjamin Lundgren, Cincinnati, OH (US)

(21) Appl. No.: 16/998,587

(22) Filed: Aug. 20, 2020

Related U.S. Application Data
(60) Provisional application No. 63/023,855, filed on May 12, 2020.

(51) Int. Cl. A61B 5/00; A61B 1/00; A61B 1/04; A61B 1/267; A61B 1/06; A61B 34/30

(52) U.S. Cl. CPC A61B 5/0071; A61B 1/00013; A61B 34/30; A61B 1/2676; A61B 1/0676; A61B 1/043

(57) ABSTRACT

The present disclosure relates to method of performing an interventional procedure using flexible probes with a compound or a composition comprising the compound, wherein the compound comprises a targeting moiety, wherein the targeting moiety targets a receptor, antigen, or antibody and a fluorescence imaging agent.

### RELATED APPLICATIONS

The present patent application claims the priority benefit of U.S. Provisional Patent Application Ser. No. 63/023,855, filed May 12, 2020 the content of which is hereby incorporated by reference in its entirety into this disclosure.

### BACKGROUND

Endoscopy is a minimally invasive or non-invasive procedure to examine the hollow interior of an organ or body cavity. Common endoscopic procedures include anoscopy, arthroscopy, bronchoscopy, colonoscopy, colposcopy, cystoscopy, esophagoscopy, gastroscopy, laparoscopy, laryngoscopy, neuroendoscopy, proctoscopy, sigmoidoscopy, and thoracoscopy. Such procedures can be performed with a flexible endoscope, an instrument that combines fiber optics and charge-coupled devices to illuminate and visualize target tissue.

Endoscopies can be stand-alone diagnostic or investigatory procedures or coupled with intervention (biopsy, ablation, resection, etc.) of target tissue. For example, an endoscopy may involve resection or removal of malignant lesions, tumors, and nodules in the ovary, kidney, lung, endometrium, breast, colon, prostate, liver, pancreas, esophagus, brain, cervix, and epithelium. While flexible endoscopes are valuable diagnostic and interventional tools, identifying cancerous lesions can be challenging due to nonspecific background from off-target tissue, limitations in localizing to an intervention site, and the heterogeneous nature of some lesions. It can therefore be advantageous to couple flexible endoscopies with identifying target tissue based on a unique molecular signature, such as overexpression of a specific protein expressed at the surface of diseased cells. The molecular contrast between normal and cancerous cells may provide an efficient method of tumor detection with fluorescently-labeled molecular targets. Examples of unique, cancer molecular signatures include folate receptor (FR) expressed in cancers of the ovary, kidney, lung, endometrium, breast, and colon; prostate-specific membrane antigen (PSMA) expressed in prostate cancer and the neovasculature of other solid tumors; cholecystokinin-2 (CCK-2) expressed in cancers of the thyroid, pancreas, lung, gastrointestinal tract, colon, and liver; carbonic anhydrase IX (CA IX) expressed in cancers of the breast, lung, kidney, colon/rectum, cervix, oral cavity, head/neck, gallbladder, liver, brain, pancreas, and gastric epithelium; glucose transporter 1 (GLUT1) expressed in cancers of the liver, pancreas, breast, esophagus, brain, kidney, lung, colon/rectum, endometrium, ovary, and cervix; fibroblast activation protein alpha (FAP-alpha) expressed in cancers of the gastrointestinal tracts, pancreas, breast, and ovary; and glutamate carboxypeptidase II (GCPII)/prostate-specific membrane antigen (PSMA) expressed in cancers of the breast and prostate.

Lung cancer, including small cell lung cancer, non-small cell lung cancer, and pulmonary nodules, is the leading cause of cancer-related death in the United States and worldwide. While early detection of lung cancer can improve patient prognosis, detection, and localization of cancerous pulmonary tissue remains challenging with existing diagnostic technology. For example, screening with low-dose computerized tomography (CT) results in 96.4% of identified nodules being false positives, while 90.4% of those require further investigation to confirm a diagnosis.

Non-invasive diagnostic procedures typically involve flexible bronchoscopy to assess pulmonary tissue and peripheral pulmonary tissue for tumors, lesions, and nodules. During bronchoscopy, a flexible endoscope is inserted in the nose or mouth of a patient. Equipped with imaging, lighting, and/or steering capabilities at its distal end, the flexible endoscope is guided through the patient's airways (bronchus). Limitations of routine flexible bronchoscopy include difficulty accessing small (less than 2 cm) and/or peripheral lesions due to the inability access beyond the subsegmental bronchi and navigate endobronchial accessory tissue. Advances in diagnostic technology include virtual bronchoscopy and electromagnetic navigation bronchoscopy.

Virtual bronchoscopy (VB) is one method of investigating pulmonary lesions, particularly those beyond the subsegmental bronchi. VB provides a simulated 3D mapping of a patient's tracheobronchial tree using pre-procedural CT imaging, therefore providing the same views and angles as real-time bronchoscopy. When VB accompanies a real-time bronchoscopy, it can provide airway information that may not be available via real-time video feed of the tracheobronchial tree due to blood, mucus, or airway swelling. However, VB is limited by the quality of the CT images and can fail to guide a practitioner through smaller bronchi. Further, it cannot account for real-time positions or provide lesion information.

Electromagnetic navigation bronchoscopy (ENB), used in conjunction with VB, utilizes an electromagnetic emitter and tracking board to emit a magnetic field around a patient's chest. A sensor is passed through the working channel of a bronchoscope to collect information on the planes, orientation, position, etc. of the tracheobronchial tree, which is analyzed by computer software. During a bronchoscopy, the coupling of ENB to VB images allows a practitioner to select from multiple views at different stages of the procedure to navigate to a target tissue. Once localized at a target, the working channel of the bronchoscope is locked and the flexible probe removed, leaving the sheath in place for subsequent interventional tools. The diagnostic yield (i.e., the proportion of patients in whom a medical technique yields a diagnosis out of the total number of patients receiving the diagnostic procedure) of ENB correlates to the presence of a bronchus sign, defined as an airway going right into a pulmonary lesion on a CT scan. In the absence of a bronchus sign on CT imaging, the diagnostic yield of ENB is drastically decreased.

A limitation of both VB and ENB is that neither provides a clear definition or delineation of a pulmonary lesion. This is particularly problematic for small lesions or peripheral lesions in which location relative to the pleura and whether the lesion is partially endobronchial or disposed entirely in the lung parenchyma is difficult to discern.

Thus, there remains a need for defining the borders and location of pulmonary lesions during bronchoscopy. Such technology would be advantageous for diagnosis, imaging, and intervention of lung cancer. It would be similarly applicable to flexible endoscopic investigation and/or intervention of the ovary, kidney, endometrium, breast, colon, prostate, thyroid, pancreas, gastrointestinal tract, liver, colon/rectum, cervix, oral cavity, head/neck, gallbladder, brain, gastric epithelium, and esophagus.

### BRIEF SUMMARY

One aspect of the present technology is a method of performing an interventional procedure, said method comprising the steps of: (a) contracting biological tissue of a human or animal subject with a compound or a composition comprising the compound, wherein the compound comprises a targeting moiety and a fluorescence imaging agent, wherein the targeting moiety targets a receptor, antigen, or antibody; (b) allowing time for the compound to distribute within the biological tissue; (c) guiding a flexible probe to the biological tissue; (d) illuminating the biological tissue; and (e) detecting the optical signal emitted by the compound.

Another aspect of the present technology is a method of performing an interventional procedure, said method comprising the steps of: (a) administering a compound or a composition comprising the compound to a human or animal subject, wherein the compound comprises a targeting moiety and a fluorescence imaging agent, wherein the targeting moiety targets a receptor, antigen, or antibody; (b) allowing time for the compound to distribute at an intervention site of the subject; (c) guiding a flexible probe to the intervention site; (d) illuminating biological tissue at the intervention site; and (e) performing intervention of the biological tissue at the intervention site.

In a further aspect, the method can be used to monitor responses to surgical procedures, chemotherapy, immunotherapy, or radiotherapy in the human or animal subject.

In another aspect, the compound is in the form of a pharmaceutically acceptable salt. In yet another aspect, the pharmaceutically acceptable salt is selected from the group consisting of sodium, potassium, calcium, magnesium, lithium, cholinate, lysinium, and ammonium.

In another aspect, the compound enhances the navigation of the flexible probe. In a further aspect, the enhanced navigation allows the flexible probe to access the final space or distance of the biological tissue or intervention site. In yet another aspect, the final space or distance is 1-3 cm from the end of the biological tissue or intervention site.

In another aspect, the optical signal is imaged in vivo. In yet another aspect, the optical signal is detected using an imaging system or imaging software.

In another aspect, the interventional procedure is non-invasive, minimally invasive, or invasive.

In another aspect, the flexible probe is a flexible endoscope, fluorescence endoscopic imaging probe, fiber scope, video scope, gastroscope, colonoscope, bronchoscope, laryngoscope, cystoscope, duodenoscope, enteroscope, ureteroscope, sigmoidoscope, enteroscope, choleodoscope, rhinolaryngoscope, angioscope, or hysteroscope. In yet another aspect, the fluorescence endoscopic imaging probe is equipped to detect wavelengths that have an absorption and emission maxima between about 400 nm and 900 nm.

In another aspect, the intervention of the biological tissue is performed using iBiopsy, iKnife, iLaser, iBurner, an electric cutting loop, a rotating blade, a curved blade, an expandable blade, dissectors with cutting blades, blunt dissectors, pinchers, an electrolyzable element, a biopsy needle, microwave ablation probe, radiofrequency ablation probe, cryo-ablation probe, or laser.

In another aspect, the biological tissue is a tumor, nodule, metastatic lesion, synchronous lesion, tumor margins, or lymph node. In yet another aspect, the tumor, metastatic lesion, synchronous lesion, tumor margins, or lymph node is in or near the lung, ovary, kidney, endometrium, breast, colon, prostate, thyroid, pancreas, gastrointestinal tract, liver, colon/rectum, cervix, oral cavity, head/neck, gallbladder, brain, gastric epithelium, or esophagus. In a further aspect, the tumor, metastatic lesion, synchronous lesion, tumor margins, or lymph node is in or near the lung of the human or animal subject and is accessed during a bronchoscopy. In yet another aspect, the bronchoscopy is non-invasive. In another aspect, the bronchoscopy can be performed manually or using robotic-assisted technology. In a further aspect, the bronchoscopy comprises biopsy, ablation, resection, incision, or cauterization.

In another aspect, the method is used in fluorescence-guided surgery or fluorescence-guided tumor resection of primary tumor, metastatic tumor, lymph node, synchronous lesions, tumor margins.

In another aspect, the method is used in fluorescence-guided ablation of primary tumor or residual tumor after the surgical removal of the primary tumor.

In yet another aspect, the method is used in fluorescence-guided ablation of metastatic tumor, lymph node, synchronous lesion, or tumor margins.

In another aspect, the targeting moiety targets a folate receptor, Glutamate carboxypeptidase II, prostate-specific membrane antigen, carbonic anhydrase IX (CA IX), Fibroblast activation protein alpha, Glucose transporter 1, or cholecystokinin-2.

In another aspect, wherein fluorescence imaging agent has an excitation and emission spectra in the near-infrared range. In a further aspect, the fluorescence imaging agent has an absorption and emission maxima between about 600 nm and 850 nm.

### BRIEF DESCRIPTION OF THE DRAWINGS

The above-mentioned and other features of this disclosure, and the manner of attaining them, will become more apparent and the disclosure itself will be better understood by reference to the following description of embodiments of the disclosure taken in conjunction with the accompanying drawings.

FIGS. 1A-1D illustrate In vivo efficacy and specificity of folate-targeted NIR imaging agent in orthotopic lung tumor model in mice. Flexible probe will be guided by the NIR signal to direct the probe to the lung tumors.

FIG. 1A is a representative of a fluorescence image of half-body of mice bearing intact orthotopic lung tumors from an IVIS image system.

FIG. 1B is a representative fluorescence image of dissected lung tissues of mice bearing orthotopic lung tumors from an IVIS image system.

FIG. 1C is a representative fluorescence image of white light image of dissected lung tissues of mice bearing orthotopic lung tumors after 2 h of administering 10 nmol of a folate-targeted NIR imaging agent from an IVIS image system.

FIG. 1D is a representative H&E staining of orthotopic lung tissues of mice bearing orthotopic tumors.

FIGS. 2A-2C illustrate in vivo efficacy and specificity of folate-targeted NIR imaging agent in orthotopic ovarian tumor model. Flexible probe will be guided by the NIR signal to direct the probe to the ovarian tumors.

FIG. 2A is a representative white light whole-body image of intact ovary of mice bearing orthotopic ovarian tumors from an IVIS image system.

FIG. 2B is a white light image of a dissected ovary of mice bearing orthotopic ovarian tumors.

FIG. 2C is a tissue biodistribution analysis of the same mice with ovarian tumors after 2 h of administering 10 nmol of folate-targeted NIR imaging agent.

FIGS. 3A-3C illustrate in vivo efficacy and specificity of PSMA-targeted NIR imaging agent in orthotopic prostate tumor model. Flexible probe will be guided by the NIR signal to direct the probe to the prostate tumors.

FIG. 3A is a representative fluorescence image from AMI image system of mice bearing orthotopic tumors 2 h after administering 10 nmol of PSMA-targeted NIR imaging agent.

FIGS. 3B and 3C illustrate issue biodistribution analysis of the same mice with (at 2 h post-injection. Note: *Primary tumor is in the prostate in Figure (c) and K=Kidneys. Note: PT=Primary Tumor, SC=Secondary Tumor, & SV=Seminal Vesicle.

### DETAILED DESCRIPTION

It is to be understood that this invention is not limited to the particular methodology, protocols, cell lines, constructs, and reagents described herein and as such may vary. It is also to be understood that the terminology used herein is for the purpose of describing particular embodiments only and is not intended to limit the scope of the present invention, which will be limited only by the appended claims.

As used herein and in the appended claims, the singular forms “a,” “an,” and “the” include plural reference unless the context clearly indicates otherwise.

Unless defined otherwise, all technical and scientific terms used herein have the same meaning as commonly understood to one of ordinary skill in the art to which this invention belongs. Although any methods, devices, and materials similar or equivalent to those described herein can be used in the practice or testing of the invention, the preferred methods, devices, and materials are now described.

All publications and patents mentioned herein are incorporated herein by reference for the purpose of describing and disclosing, for example, the constructs and methodologies that are described in the publications, which might be used in connection with the presently described invention. The publications discussed herein are provided solely for their disclosure prior to the filing date of the present application. Nothing herein is to be construed as an admission that the inventors are not entitled to antedate such disclosure by virtue of prior invention or for any other reason.

The terms “functional group”, “active moiety”, “activating group”, “leaving group”, “reactive site”, “chemically reactive group” and “chemically reactive moiety” are used in the art and herein to refer to distinct, definable portions or units of a molecule. The terms are somewhat synonymous in the chemical arts and are used herein to indicate the portions of molecules that perform some function or activity and are reactive with other molecules.

The term “amino acid” refers to naturally occurring and non-naturally occurring amino acids, as well as amino acid analogs and amino acid mimetics that function in a manner similar to the naturally occurring amino acids. Naturally encoded amino acids are the 20 common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) and pyrolysine and selenocysteine. Amino acid analogs refer to compounds that have the same basic chemical structure as a naturally occurring amino acid, i.e., an a carbon that is bound to a hydrogen, a carboxyl group, an amino group, and an R group, such as, homoserine, norleucine, methionine sulfoxide, methionine methyl sulfonium. Such analogs have modified R groups (such as norleucine) or modified peptide backbones, but retain the same basic chemical structure as a naturally occurring amino acid.

Amino acids may be referred to herein by either their commonly known three letter symbols or by the one-letter symbols recommended by the IUPAC-IUB Biochemical Nomenclature Commission.

In some aspects of the invention, the compounds can be used for image-guided surgery, tumor imaging, lymph node imaging, inflammatory diseases, atherosclerosis, infection diseases, forensic applications, mineral applications, dental, gel staining, DNA sequencing, nerve staining, or plastic surgery.

In some aspects of the invention, the compound may be incorporated into targeting moieties which may include a protein or polypeptide, such as an antibody, or biologically active fragment thereof, preferably a monoclonal antibody, small molecules, aptamers, DNA, or RNA. The supplemental fluorescing targeting construct(s) used in practice of the disclosed method may also be or comprise polyclonal or monoclonal antibodies tagged with a fluorophore. The term “antibody” as used in this disclosure includes intact molecules as well as functional fragments thereof, such as Fab, F(ab′)2, and Fv that are capable of binding the epitopic determinant. Methods of making these fragments are known in the art. (See, for example, Harlow & Lane, Antibodies: A Laboratory Manual, Cold Spring Harbor Laboratory, N.Y., 1988, incorporated herein by reference). As used in this disclosure, the term “epitope” means any antigenic determinant on an antigen to which the paratope of an antibody binds. Epitopic determinants usually consist of chemically active surface groupings of molecules such as amino acids or sugar side chains. Usually, they have specific three-dimensional structural characteristics, as well as specific charge characteristics.

In some aspects, a compound may be incorporated or used with other fluorescing targeting constructs (e.g., antibodies, or biologically active fragments thereof, having attached fluorophores) that bind to other receptors or antigens on the tumor or tissue (e.g., a site of atherosclerosis, infection, cardiovascular disease, neurodegenerative disease, immunologic disease, autoimmune disease, respiratory disease, metabolic disease, inherited disease, infectious disease, bone disease, and environmental disease or the like) to be imaged. Any additional targeting moiety that specifically targets the tumor or specific site on the tissue may be used provided that it is specific for the site to be monitored. The purpose of the additional fluorescing targeting construct is to increase the intensity of fluorescence at the site to be monitored, thereby aiding in the detection of diseased or abnormal tissue in the body part. For example, a given tumor may have numerous markers. In addition to the compounds of the present disclosure, a cocktail of fluorescent moieties is provided, which are specific for that given tumor such that the signal emanating from the tumor is generated by more than one compound or fluorescent moiety that has targeted and localized to the tumor site of interest.

In practice, the skilled person would administer a compound of the present disclosure either alone or as part of a cocktail of targeting detectable moieties and allow these compounds and targeting moieties to bind to and/or be taken up by any target tissue that may be present at the site under investigation and then provide a supply of the light source. Typically, the compounds of the present disclosure and any additional targeting moieties will be administered prior to surgery or a minimally invasive or non-invasive procedure for a time and in compositions that allow the fluorescent compounds of the present disclosure as well as any additional fluorescent constructs to be taken up by the target tissue.

Those of skill in the art will be able to devise combinations of successively administered fluorescing targeting constructs, each of which specifically binds to the target site. It is preferable that all of the fluorescing targeting constructs used in such cocktails to identify the target tissue comprise fluorophores that fluoresce within the same wavelength band or at the same wavelength as does the compound of the present disclosure (e.g., a fluorescing sensitive to a near-infrared wavelength of light in the compounds of the present disclosure) to minimize the number of different light sources that need to be employed to excite simultaneous fluorescence from all of the different targeting constructs used in practice of the disclosure method. However, it is contemplated that the additional targeting moieties other than the compounds of the present disclosure may fluoresce in response to the irradiating light at a different color (i.e., has a different wavelength) than that from the fluorescent compounds of the present disclosure. The difference in the colors of the fluorescence emanating from the compounds of the present disclosure and those of the additional targeting compounds may aid the observer in determining the location and size of the diseased or target tissue. In some examples, it may be desirable to include fluorophores in targeting constructs targeted to normal tissue and the compounds of the present disclosure targeted to diseased tissue such that the contrast between the diseased tissue and normal tissue is further enhanced to further aid the observer in determining the location and size of the diseased tissue. The use of such additional fluorophores and targeting agents in addition to the compounds of the present disclosure provides the advantage that any natural fluorescence emanating from normal tissue is obscured by the fluorescence emanating from fluorophore(s) in supplemental targeting constructs targeted to the normal tissue in the body part. The greater the difference in color between the fluorescence emanating from normal and target tissue, the easier it is for the observer to visualize the outlines and size of the target tissue. For instance, targeting a fluorescing targeting construct comprising a fluorophore producing infrared light from the compounds of the present disclosure to the target tissue (i.e., abnormal tissue) and a fluorophore producing green light to healthy tissue aids the observer in distinguishing the target tissue from the normal tissue. Those of skill in the art can readily select a combination of fluorophores that present a distinct visual color contrast.

The spectrum of light used in the practice of the disclosed method is selected to contain at least one wavelength that corresponds to the predominate excitation wavelength of the targeting construct, or of a biologically compatible fluorescing moiety contained within the targeting construct.

However, when a combination of targeting ligands that fluoresce at different wavelengths is used in the practice of the disclosure, the spectrum of the excitation light must be broad enough to provide at least one excitation wavelength for each of the fluorophores used. For example, it is particularly beneficial when fluorophores of different colors are selected to distinguish normal from diseased tissue, that the excitation spectrum of the light(s) includes excitation wavelengths for the fluorophores targeted to normal and target tissue.

In one aspect of the present disclosure, the compounds are used to identify a target cell type in a biological sample by contacting the biological sample with such compounds for a time and under conditions that allow for binding of the compound to at least one cell of the target cell type. The bound compound is then optically detected, such that the presence of fluorescence of the near-infrared wavelength emanating from the bound, targeted compound of the present disclosure indicated that the target cell type is present in the biological sample. This method thus provides an image of the targeted cell type in the tissue being assessed. Most preferably, the targeted cell type is a tumor cell or a lymph node to which a tumor cell has spread.

These methods advantageously provide an improved method of performing image-guided surgery on a subject as the administration of a composition comprising the compound of the disclosure under conditions and for a time sufficient for said compound to accumulate at a given surgical site will assist a surgeon in visualizing the tissue to be removed. Preferably the tissue is a tumor tissue, and illuminating the compound that has been taken up by the tissue facilitates visualization of the tumor by the near-infrared fluorescence of the compound using infrared light. With the aid of the visualization facilitated by the targeting of the compound of the disclosure to the site of the tumor, surgical resection of the areas that fluoresce upon excitation by infrared light allows an improved and accurate removal of even small tumors.

One aspect of the present disclosure provides methods of performing an interventional procedure, said method comprising the steps of: (a) contracting biological tissue of a human or animal subject with a compound, a pharmaceutically acceptable salt of the compound, a composition comprising the compound or pharmaceutically acceptable salt of the compound, wherein the compound comprises a targeting moiety and a fluorescence imaging agent, wherein the targeting moiety targets a receptor, antigen, or antibody; (b) allowing time for the compound to distribute within the biological tissue; (c) guiding a flexible probe to the biological tissue; (d) illuminating the biological tissue; and (e) detecting the optical signal emitted by the compound. This method can be used to monitor responses to surgical procedures, chemotherapy, immunotherapy, or radiotherapy in the human or animal subject.

Another aspect of the present disclosure provides a method of performing an interventional procedure, said method comprising the steps of: (a) administering a compound or a composition comprising the compound to a human or animal subject, wherein the compound comprises a targeting moiety and a fluorescence imaging agent, wherein the targeting moiety targets a receptor, antigen, or antibody; (b) allowing time for the compound to distribute at an intervention site of the subject; (c) guiding a flexible probe to the intervention site; (d) illuminating biological tissue at the intervention site; and (e) performing intervention of the biological tissue at the intervention site.

In some aspects, the interventional procedure is non-invasive, minimally invasive, or invasive. The term “invasive interventional procedure”, as used herein, refers to interventional procedures requiring an incision (i.e., breaks the skin in some way) to reach an intervention site, and can include surgery. The term “minimally invasive interventional”, as used herein, employs techniques that limit the size of incisions needed and so lessen wound healing time, associated pain and risk of infection, and can include surgery. The term “non-invasive interventional procedure”, as used herein, refers to interventional procedures that do not require an incision and do not break the skin to reach an intervention site. Non-invasive interventional procedures can include manipulation of tissue at the intervention site.

In some aspects, the compound is in the form of a pharmaceutically acceptable salt.

The pharmaceutically acceptable salt can be selected from the group consisting of sodium, potassium, calcium, magnesium, lithium, cholinate, lysinium, or ammonium.

The inventors have surprisingly discovered that the use of the compound enhanced the navigation of the flexible probe. Specifically, the compound proves particularly useful in accessing the final space or distance of the biological tissue or intervention site, such as 1 cm from the end of the biological tissue or intervention site, alternatively 2 cm from the end of the biological tissue or intervention site, alternatively 3 cm from the end of the biological tissue or intervention site.

In one aspect, the optical image is imaged in vivo and can be detected using an imaging system or imaging software.

In one aspect, the flexible probe is a flexible endoscope, fluorescence endoscopic imaging probe, fiber scope, video scope, gastroscope, colonoscope, bronchoscope, laryngoscope, cystoscope, duodenoscope, enteroscope, ureteroscope, sigmoidoscope, enteroscope, choleodoscope, rhinolaryngoscope, angioscope, or hysteroscope. In other aspects, the fluorescence endoscopic imaging probe is equipped to detect wavelengths that have an absorption and emission maxima between about 400 nm and 900 nm.

In some aspects, the interventional procedure may involve biopsy, ablation, resection, incision, cutting, and/or cauterization of target tissue within a subject using a near-infrared enabled flexible endoscope. In some aspects, the target tissue can be a tumor requiring biopsy, ablation, resection, incision, and/or cauterization from a tumor bed. Such methods can be carried out using tools and techniques known in the art such as, but not limited to iBiopsy (an “intelligent” biopsy device comprising a needle sheath that enables real-time (i.e., during an interventional procedure) spectral measurement and histology of tissue), iKnife (an “intelligent” Knife that uses rapid evaporative ionization mass spectrometry (REIMS) for real-time histology of aerosolized tissue), iLaser probe, iBurner, and flexible endoscopes equipped with an electric cutting loop, a rotating blade, a curved blade, an expandable blade, dissectors with cutting blades, blunt dissectors, pinchers, an electrolyzable element for cauterization or resection, a blade, a scalpel, a biopsy needle, a microwave ablation probe, a radiofrequency ablation probe, a cryo-ablation probe, laser, etc. In some aspects, such procedures can be performed using Confocal Laser Endomicroscopy.

In certain aspects, image-guided biopsy, ablation, resection, incision, cutting, and/or cauterization can be performed on primary lung tumor nodules, metastatic lung lesions, and regional metastatic lung lymph nodes. Using the compounds disclosed herein in combination with a near-infrared enabled flexible endoscope, a practitioner can ensure the endoscope attachment (e.g., biopsy needle, ablation probe, cauterization probe, etc.) is correctly positioned at an intervention site, such as a tumor nodule or bed, before initiating treatment. In some aspects, image-guided biopsy, ablation, resection, incision, cutting, and/or cauterization of lung tumor nodules, metastatic lung lesions, and regional metastatic lung lymph nodes can be performed during a bronchoscopy. In certain aspects, image-guided cauterization of a tumor bed can be performed in conjunction with an ablation treatment to ensure clear margins after tumor removal. In some aspects, these methods can employ iSite or iVision.

Accordingly, the diseased tissue (and bound or taken-up targeting construct) is “exposed” to the excitation light (e.g., endoscopic delivery of the light to an interior location). The disclosure of these methods of imaging is particularly suited to in vivo detection of diseased tissue located at an interior site in the subject, such as within a natural body cavity or a surgically created opening, where the diseased tissue is “in plain view” (i.e., exposed to the human eye) to facilitate a procedure of biopsy or surgical excision of the area that has been highlighted by uptake of the compounds of the present disclosure. As the precise location and/or surface area of the diseased or inflamed tissue are readily determined by the uptake of the compounds of the present disclosure, the methods employing the compounds of the present disclosure provide a valuable guide to pathologists, immunologists, technicians and surgeons alike, who needs to “see” in real-time the exact outlines, size, etc., of the mass of the inflamed areas for diagnosis and imaging, and if necessary, surgery. If the putative diseased site is a natural body cavity or surgically produced interior site, an endoscopic attachment can be used to deliver the excitation light to the site, to receive fluorescence emanating from the site within a body cavity, and to aid in the formation of a direct image of the fluorescence from the diseased tissue. For example, a lens in the endoscopic attachment can be used to focus the detected fluorescence as an aid in the formation of the image.

In some aspects, the biological tissue is a tumor, nodule, metastatic lesion, synchronous lesion, tumor margins, or lymph node. In another aspect, the tumor, metastatic lesion, synchronous lesion, tumor margins, or lymph node is in or near the lung, ovary, kidney, endometrium, breast, colon, prostate, thyroid, pancreas, gastrointestinal tract, liver, colon/rectum, cervix, oral cavity, head/neck, gallbladder, brain, gastric epithelium, or esophagus.

In another aspect, when the biological tissue (i.e., tumor, metastatic lesion, synchronous lesion, tumor margins, or lymph node) is in or near the lung of the human or animal subject, it can be accessed during a bronchoscopy. In a further aspect, the bronchoscopy is non-invasive. In yet another aspect, the bronchoscopy can be performed manually or using robotic-assisted technology. The bronchoscopy also may comprise biopsy, ablation, resection, incision, or cauterization. In some aspects, such procedures can be performed using a near-infrared enabled flexible endoscope inserted through the nose or mouth of a subject and navigated to target tissue using the compounds disclosed herein, during a bronchoscopy. It is contemplated such methods can be used to diagnose a disease in a subject.

In some aspects, the method is used in fluorescence-guided surgery or fluorescence-guided tumor resection of primary tumor, metastatic tumor, lymph node, synchronous lesions, tumor margins. In another aspect, the method is used in fluorescence-guided ablation of primary tumor or residual tumor after the surgical removal of the primary tumor. In yet another aspect, the method is used in fluorescence-guided ablation of metastatic tumor, lymph node, synchronous lesion, or tumor margins.

In some aspects, the targeting moiety targets a folate receptor, Glutamate carboxypeptidase II, prostate-specific membrane antigen, carbonic anhydrase IX (CA IX), Fibroblast activation protein alpha, Glucose transporter 1, or cholecystokinin-2. In some aspects, the targeting moiety is conjugated to an amino acid linking group. In another aspect, the targeting moiety is selected from a group comprising of a pteroyl ligand, PSMA-targeting compound, or CA IX-targeted molecule conjugated to an amino acid linking group.

In some aspects, the imaging agent is detectable outside the visible light spectrum. In some aspects, the imaging agent is greater than the visible light spectrum. In some aspects, fluorescence imaging agent has an excitation and emission spectra in the near-infrared range. The fluorescence imaging agent may have an absorption and emission maxima between about 600 nm and 1000 nm, alternatively between about 600 nm and 850 nm, alternatively between about 650 nm and 850 nm.

Light having a wavelength range from 600 nm and 850 nm lies within the near-infrared range of the spectrum, in contrast to visible light, which lies within the range from about 400 nm to about 500 nm. Therefore, the excitation light used in the practice of the disclosed methods will contain at least one wavelength of light to illuminates the tissue at the infrared wavelength to excite the compounds so that the fluorescence obtained from the area having uptake of the compounds of the present disclosure is clearly visible and distinct from the auto-fluorescence of the surrounding tissue. The excitation light may be monochromatic or polychromatic. In this manner, the compounds of the present disclosure are advantageous as they eliminate the need for the use of filtering mechanisms that would be used to obtain a desired diagnostic image if the fluorescent probe is one that fluoresces at wavelengths below about 600 nm. In this manner, the compounds of the present disclosure avoid obscured diagnostic images that are produced as a result of excitation light of wavelengths that would be reflected from healthy tissue and cause loss of resolution of the fluorescent image.

In some aspects, a single type of fluorescent moiety is relied upon for generating fluorescence emanating from the irradiated body part. In other aspects, it is contemplated that a plurality of (i.e., two, three, four, or more) targeting constructs are used to obtain a diagnostic image. When a combination of targeting ligands that fluoresce at different wavelengths is used in the practice of the disclosure, the spectrum of the excitation light must be broad enough to provide at least one excitation wavelength for each of the fluorophores used.

In some aspects, the cells, tissue, and/or tumor being detected are more than 5 mm below the skin of a subject. Alternatively, the cells, tissue, and/or tumor being detected are more than 6 mm, 7 mm, 8 mm, 9 mm, or 10 mm below the subject's skin. In some aspects, the subject is a mammal. In other aspects, the mammal is a human.

In some aspects, the tumor has a volume of at least 1000 mm³. In some aspects, the volume of the tumor is less than 1000 mm³. In some aspects, the volume of the tumor is less than 950 mm³. In some aspects, the volume of the tumor is less than 900 mm³. In some aspects, the volume of the tumor is less than 850 mm³. In some aspects, the volume of the tumor is less than 800 mm³. In some aspects, the volume of the tumor is less than 750 mm³. In some aspects, the volume of the tumor is less than 700 mm³. In some aspects, the volume of the tumor is less than 650 mm³. In some aspects, the volume of the tumor is less than 600 mm³. In some aspects, the volume of the tumor is less than 550 mm³. In some aspects, the volume of the tumor is less than 500 mm³. In some aspects, the volume of the tumor is less than 450 mm³. In some aspects, the volume of the tumor is less than 400 mm³. In some aspects, the volume of the tumor is less than 350 mm³. In some aspects, the volume of the tumor is less than 300 mm³. In some aspects, the volume of the tumor is less than 250 mm³. In some aspects, the volume of the tumor is less than 200 mm³. In some aspects, the volume of the tumor is less than 150 mm³. In some aspects, the volume of the tumor is less than 100 mm³. In one aspect, the volume of the tumor is at least 75 mm³. In another aspect, the volume of the tumor is less than 75 mm³. In another aspect, the volume of the tumor is less than 70 mm³. In another aspect, the volume of the tumor is less than 65 mm³. In another aspect, the volume of the tumor is less than 60 mm³. In another aspect, the volume of the tumor is less than 55 mm³. In one aspect, the volume of the tumor is at least 50 mm³. In another aspect, the tumor is less than 50 mm³. In another aspect, the volume of the tumor is less than 45 mm³. In other aspects, the volume of the tumor is less than 40 mm³. In another embodiment, the volume of the tumor is less than 35 mm³. In still another aspect, the volume of the tumor is less than 30 mm³. In another aspect, the volume of the tumor is less than 25 mm³. In still another aspect, the volume of the tumor is less than 20 mm³. In another aspect, the volume of the tumor is less than 15 mm³. In still another aspect, the volume of the tumor is less than 10 mm³. In still another aspect, the volume of the tumor is less than 12 mm³. In still another aspect, the volume of the tumor is less than 9 mm³. In still another aspect, the volume of the tumor is less than 8 mm³. In still another aspect, the volume of the tumor is less than 7 mm³. In still another aspect, the volume of the tumor is less than 6 mm³. In still another aspect, the volume of the tumor is less than 5 mm³.

In some aspects, these methods detect tumors less than 5 mm. In other aspects, the methods detect tumors less than 4 mm. In some aspects, the methods herein detect tumors less than 3 mm. In another aspect, the tumor has a length of at least 6 mm. In still another aspect, the tumor has a length of at least 7 mm. In yet another aspect, the tumor has a length of at least 8 mm. In another aspect, the tumor has a length of at least 9 mm. In still another aspect, the tumor has a length of at least 10 mm. In yet another aspect, the tumor has a length of at least 11 mm. In a further aspect, the tumor has a length of at least 12 mm. In still a further aspect, the tumor has a length of at least 13 mm. In still a further aspect, the tumor has a length of at least 14 mm. In another aspect, the tumor has a length of at least 15 mm. In yet another aspect, the tumor has a length of at least 16 mm. In still another aspect, the tumor has a length of at least 17 mm. In a further aspect, the tumor has a length of at least 18 mm. In yet a further aspect, the tumor has a length of at least 19 mm. In still a further aspect, the tumor has a length of at least 20 mm. In another aspect, the tumor has a length of at least 21 mm. In still another aspect, the tumor has a length of at least 22 mm. In yet another aspect, the tumor has a length of at least 23 mm. In a further aspect, the tumor has a length of at least 24 mm. In still a further aspect, the tumor has a length of at least 25 mm. In yet a further aspect, the tumor has a length of at least 30 mm.

It is further contemplated that the compounds disclosed herein can also be administered to a subject to perform optical imaging of a previous surgical or intervention site to monitor tumor progression or regression or response to treatment or surgery. Such monitoring could be performed with a near-infrared enabled flexible endoscope. When lung tissue is monitored, optical imaging can be performed during a bronchoscopy.

The disease or abnormal state detected by the disclosed method can be any type characterized by the presence of a known target tissue for which a specific binding ligand is known. It is contemplated that the target tissue may be characterized by cells that produce either a surface antigen for which a binding ligand is known or an intracellular marker (i.e., antigen) since many targeting constructs penetrate the cell membrane. Representative diseases include such various conditions as different types of tumors, bacterial, fungal and viral infections, and the like. As used herein, “diseased” or “abnormal” tissue includes precancerous conditions, necrotic or ischemic tissue, and tissue associated with precancerous states as well as cancer and the like.

It should be understood that in any of the methods of the disclosure, the compounds of the present disclosure may be administered before the surgical incision takes place or even after the surgical cavity and site of the tumor have been revealed by the surgery.

It is contemplated that the diagnostic or imaging methods of the present disclosure allow the surgeon/practitioner to contemporaneously see/view/visualize diseased or abnormal tissue through a surgical opening to facilitate a procedure of biopsy or surgical excision. As the location and/or surface area of the diseased tissue are readily determined by the diagnostic procedure of the disclosure employing the compounds described herein, the disclosure method is a valuable guide to the surgeon, who needs to know the exact outlines, size, etc. of the mass, for example, for resection as the surgery proceeds. In particular, it is noted that the compounds of the disclosure fluorescence in the near-infrared range to a greater intensity than those previously described. As such, advantageously, it is contemplated that less of the compound will be needed to achieve diagnostic imaging. In addition, the compounds of the present disclosure penetrate deep into the tumor, and hence the disclosure advantageously allows a greater accuracy that the tumor has been removed.

The present disclosure provides methods for utilizing a diagnostic procedure during surgery in a subject in need thereof by administering to the subject a composition comprising a compound of the present disclosure and irradiating an in vivo body part of the subject containing diseased tissue with light having at least one excitation wavelength in the range from about 600 nm to about 850 nm, directly viewing fluorescence emanating from a targeting construct administered to the subject that has specifically bound to and/or been taken up by the diseased tissue in the body part, wherein the targeting construct fluoresces in response to the at least one excitation wavelength, determining the location and/or surface area of the diseased tissue in the subject, and removing at least a portion of the tumor tissue.

The compounds and compositions used in the disclosed methods are administered in an “effective amount.” An effective amount is the quantity of a targeting construct necessary to aid in direct visualization of any target tissue located in the body part under investigation in a subject. A “subject” is contemplated to include any mammal, such as a domesticated pet, farm animal, or zoo animal, but preferably is a human. Amounts effective for diagnostic use will, of course, depend on the size and location of the body part to be investigated, the affinity of the targeting construct for the target tissue, the type of target tissue, as well as the route of administration. Local administration of the targeting construct will typically require a smaller dosage than any mode of systemic administration, although the local concentration of the targeting construct may, in some cases, be higher following local administration than can be achieved with safety upon systemic administration.

An effective amount of the conjugate compound to be administered will be dependent on the patient's condition including surgical conditions such as blood loss, the disease state being treated, the molecular weight of the conjugate, its route of administration and tissue distribution, and the possibility of co-usage with therapeutic treatments such as radiation therapy, or chemotherapies radiation therapy. The effective amount to be administered to a patient is based on body surface area, patient weight, and physician assessment of patient condition. In various exemplary embodiments, an effective dose amount may be done with or without an excipient/carrier, including but not limited to saline. Since individual subjects may present a wide variation in severity of symptoms and each targeting construct has its unique diagnostic characteristics, including, affinity of the targeting construct for the target, rate of clearance of the targeting construct by bodily processes, the properties of the fluorophore contained therein, and the like, the skilled practitioner will weigh the factors and vary the dosages accordingly.

It will be apparent to those skilled in the art that various changes may be made in the disclosure without departing from the spirit and scope thereof, and therefore, the disclosure encompasses embodiments in addition to those specifically disclosed in the specification, but only as indicated in the appended claims.

The examples that follow are merely provided for the purpose of illustrating particular embodiments of the disclosure and are not intended to be limiting to the scope of the appended claims. As discussed herein, particular features of the disclosed compounds and methods can be modified in various ways that are not necessary to the operability or advantages they provide. For example, the compounds can incorporate a variety of amino acids and amino acid derivatives, as well as targeting ligands depending on the particular use for which the compound will be employed. One of skill in the art will appreciate that such modifications are encompassed within the scope of the appended claims.

### EXAMPLE

### Interventional Image-Guided Surgery with Tumor-Targeted Dyes

Whole-body Imaging & Tissue biodistribution: For orthotopic tumors, 2 x 10⁵ human ovarian cancer or prostate cancer cells/mouse were surgically implanted in the ovary or prostate of seven-week-old female or male SCID mice. For orthotopic lung tumors, 2×10⁵ human lung cancer cells/mouse were intravenously injected in the tail vein of seven-week-old female SCID mice. Briefly, for orthotopic prostate tumors, SCID mice were given 1-5% isoflurane for anesthesia and subcutaneous injection of 5 mg/kg meloxicam preoperatively for analgesia. The mice were placed dorsal side up and washed above the prostate with a chlorhexidine scrub to ensure a sterile area for an incision. After an insertion was made using a scalpel through the skin, the peritoneal lining was lifted to make a small incision using a scissor and widened using forceps. Dorsal lobes were exteriorized and gently stabilized with a wet (PBS) cotton swab. 22Rv1 cells (in 10 μL of 10% HC-matrigel) were injected into the prostate using a 28-gage needle. After placing the prostate back into the peritoneum, the abdominal wall was sutured, the body wall was closed using 3-0 or 4-0 vicryl, and the skin was closed using staples. Animals were monitored until use them for the studies. Similar procedures were followed for orthotopic ovarian tumor implantation.

After one month, the animals were administered with either folate-targeted or PSMA-targeted NIR imaging agent (10 nmol in 100 μL saline per mouse), euthanized after 2 h by CO2 asphyxiation, and imaged using AMI image system. For whole-body imaging and biodistribution studies, animals were euthanized after 2 h of administration of tumor-targeted NIR imaging agent by CO2 asphyxiation. Following whole-body imaging, animals were dissected, and selected tissues were analyzed for fluorescence activity using IVIS or AMI image system, and ROI of the tissues were calculated using Living Image 4.0 software or AMI View Image Analysis Software.

For immunohistopathology (IHC) studies, selected tumor tissues (lung, ovarian, or prostate) were collected into vials containing 4% formalin. Formalin-fixed tissues were sectioned into 10 μm thick sections and mounted onto Superfrost Plus™ slides (Fisher Scientific, Pittsburgh Pa.). After staining the slides with H&E, IHC analysis of the tissues was conducted.

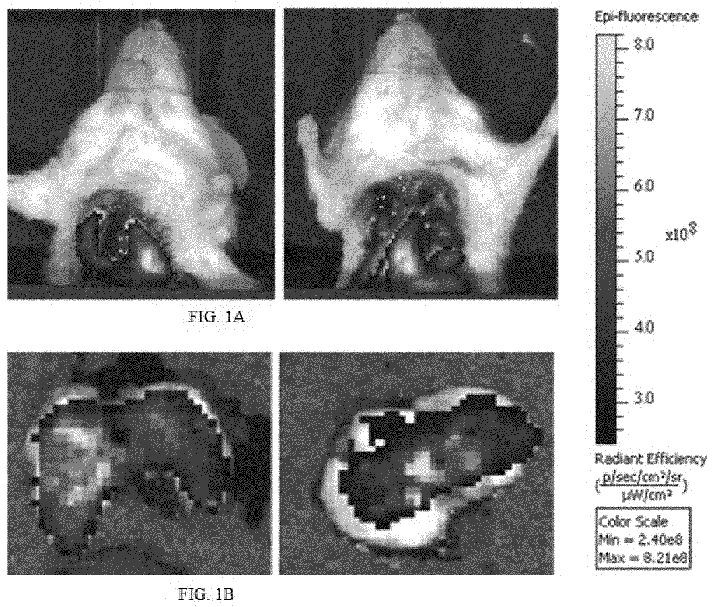

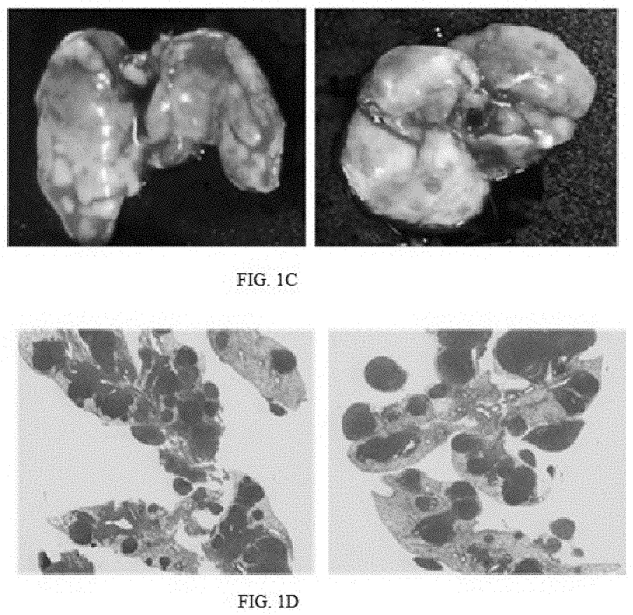

FIGS. 1A-D illustrate in vivo efficacy and specificity of folate-targeted NIR imaging agent in orthotopic lung tumor model. Representative images from IVIS image system showing mice bearing orthotopic lung tumors in (FIG. 1A) fluorescence imaging of half-body of mice with intact lung tumors, (FIG. 1B) fluorescence imaging of dissected lung tissues with tumors, and (FIG. 1C) white light image of dissected lung tissues with tumors after 2 h of administering 10 nmol of folate-targeted NIR imaging agent. FIG. 1D is a representative H&E staining of orthotopic lung tissues with orthotopic tumors. Flexible probe will be guided by the NIR signal to direct the probe to the lung tumors.

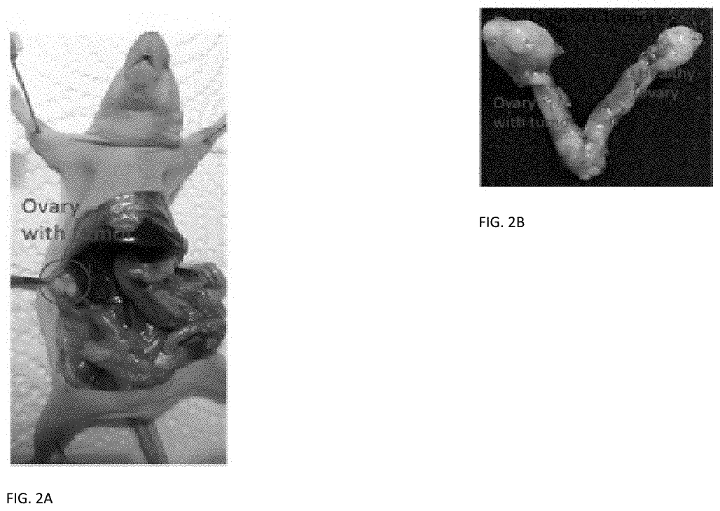

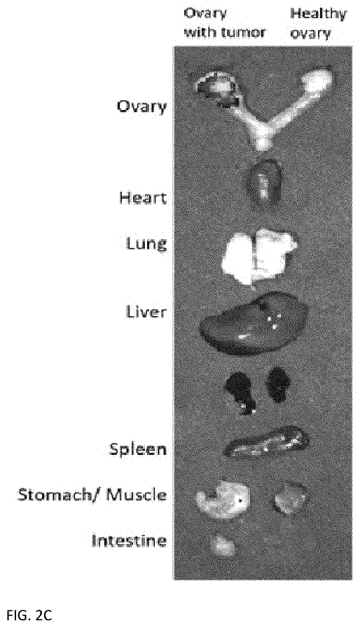

FIGS. 2A-2C illustrate in vivo efficacy and specificity of folate-targeted NIR imaging agent in orthotopic ovarian tumor model. Representative fluorescence images from IVIS image system showing mice bearing orthotopic ovarian tumors in (FIG. 2A) white light whole-body imaging with intact ovary with tumors, (FIG. 2B) white light image of a dissected ovary, and (FIG. 2C) Tissue biodistribution analysis of the same mice with ovarian tumors after 2 h of administering 10 nmol of folate-targeted NIR imaging agent. Flexible probe will be guided by the NIR signal to direct the probe to the ovarian tumors.

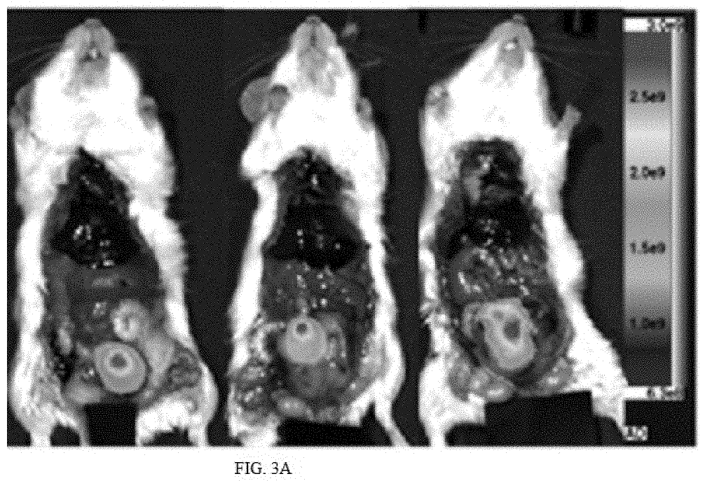

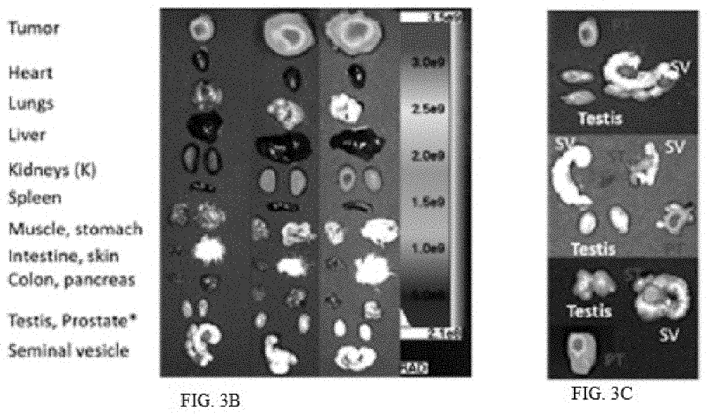

FIGS. 3A-3C illustrate in vivo efficacy and specificity of PSMA-targeted NIR imaging agent in orthotopic prostate tumor model. Representative fluorescence images from AMI image system showing mice bearing (FIG. 3A) orthotopic tumors 2 h after administering 10 nmol of PSMA-targeted NIR imaging agent. Tissue biodistribution analysis of the same mice with (FIG. 3B and 3C) at 2 h post-injection. Note: Primary tumor is in the prostate in Figure (c) and K=Kidneys. Note: PT=Primary Tumor, SC=Secondary Tumor, & SV=Seminal Vesicle. Flexible probe will be guided by the NIR signal to direct the probe to the prostate tumors.

While the present invention has been described with reference to certain aspects, it will be understood by those skilled in the art that various changes may be made and equivalents may be substituted without departing from the scope of the present invention. In addition, many modifications may be made to adapt a particular situation or material to the teachings of the present invention without departing from its scope. Therefore, it is intended that the present invention not be limited to the particular embodiment disclosed, but that the present invention will include all aspects falling within the scope of the appended claims.

1. A method of performing an interventional procedure, said method comprising the steps of:
(a) contracting biological tissue of a human or animal subject with a compound or a composition comprising a compound or administering a compound or a composition comprising a compound to a human or animal subject, wherein the compound comprises a targeting moiety and a fluorescence imaging agent, wherein the targeting moiety targets a receptor, antigen, or antibody,
(b) allowing time for the compound to distribute within the biological tissue;
(c) guiding a flexible probe to the biological tissue;
(d) illuminating the biological tissue; and
(e) detecting the optical signal emitted by the compound or performing intervention of the biological tissue at the intervention site.

2. The method of claim 1, wherein the method can be used to monitor responses to surgical procedures, chemotherapy, immunotherapy, or radiotherapy in the human or animal subject.

3. (canceled)

4. The method of claim 1, wherein the compound is in the form of a pharmaceutically acceptable salt.

5. The method of claim 4, wherein the pharmaceutically acceptable salt is selected from the group consisting of sodium, potassium, calcium, magnesium, lithium, cholinate, lysinium, or ammonium.

6. The method of claim 1, wherein the compound enhances the navigation of the flexible probe.

7. The method of claim 6, wherein the enhanced navigation allows the flexible probe to access the final space or distance of the biological tissue or intervention site.

8. The method of claim 7, wherein the final space or distance is 1-3 cm from the end of the biological tissue or intervention site.

9. The method of claim 1, wherein the optical signal is imaged in vivo.

10. The method of claim 1, wherein the optical signal is detected using an imaging system or imaging software.

11. The method of claim 1, wherein the interventional procedure is non-invasive, minimally invasive, or invasive.

12. The method of claim 1, wherein the flexible probe is a flexible endoscope, fluorescence endoscopic imaging probe, fiber scope, video scope, gastroscope, colonoscope, bronchoscope, laryngoscope, cystoscope, duodenoscope, enteroscope, ureteroscope, sigmoidoscope, enteroscope, choleodoscope, rhinolaryngoscope, angioscope, or hysteroscope.

13. The method of claim 12, wherein the fluorescence endoscopic imaging probe is equipped to detect wavelengths that have an absorption and emission maxima between about 400 nm and 900 nm.

14. The method of claim 1, wherein intervention of the biological tissue is performed using iBiopsy, iKnife, iLaser, iBurner, an electric cutting loop, a rotating blade, a curved blade, an expandable blade, dissectors with cutting blades, blunt dissectors, pinchers, an electrolyzable element, a biopsy needle, microwave ablation probe, radiofrequency ablation probe, cryo-ablation probe, or laser.

15. The method of claim 1, wherein the biological tissue is a tumor, nodule, metastatic lesion, synchronous lesion, tumor margins, or lymph node.

16. The method of claim 15, wherein the tumor, metastatic lesion, synchronous lesion, tumor margins, or lymph node is in or near the lung, ovary, kidney, endometrium, breast, colon, prostate, thyroid, pancreas, gastrointestinal tract, liver, colon/rectum, cervix, oral cavity, head/neck, gallbladder, brain, gastric epithelium, or esophagus.

17. The method of claim 15, wherein the tumor, metastatic lesion, synchronous lesion, tumor margins, or lymph node is in or near the lung of the human or animal subject and is accessed during a bronchoscopy.

18. The method of claim 17, wherein the bronchoscopy is non-invasive.

19. The method of claim 17, wherein the bronchoscopy can be performed manually or using robotic-assisted technology.

20. The method of claim 17, wherein the bronchoscopy comprises biopsy, ablation, resection, incision, or cauterization.

21. The method of claim 1, wherein the method is used in fluorescence-guided surgery or fluorescence-guided tumor resection of primary tumor, metastatic tumor, lymph node, synchronous lesions, tumor margins.

22. The method of claim 1, wherein the method is used in fluorescence-guided ablation of primary tumor or residual tumor after the surgical removal of the primary tumor.

23. The method of claim 1, wherein the method is used in fluorescence-guided ablation of metastatic tumor, lymph node, synchronous lesion, or tumor margins.

24. The method of claim 1, wherein the targeting moiety targets a folate receptor, Glutamate carboxypeptidase II, prostate-specific membrane antigen, carbonic anhydrase IX (CA IX), Fibroblast activation protein alpha, Glucose transporter 1, or cholecystokinin-2.
